(19) United States
(12) Patent Application Publication
(10) Pub. No.: US 2014/0048431 A1
(43) Pub. Date: Feb. 20, 2014

(54) SURGICAL PACK

(71) Applicant: Duncan Young, Leeds (GB)

(72) Inventor: Duncan Young, Leeds (GB)

(73) Assignee: DEPUY (IRELAND), COUNTY CORK (IE)

(21) Appl. No.: 14/110,305

(22) PCT Filed: Feb. 23, 2012
(86) PCT No.: PCT/GB12/50412

§ 371 Date: Nov. 4, 2013

(30) Foreign Application Priority Data
Apr. 7, 2011 (GB) 1105910.2

(51) Int. Cl. A61B 19/02; A61L 2/00

(52) U.S. Cl. CPC A61B 19/026; A61L 2/00
USPC 206/363

(57) ABSTRACT

A surgical pack, method of providing a surgical pack and method of use are described. The surgical pack comprises (100) a container (102) having an opening (116), at least one item of surgical equipment in the container and a closure (104) which shuts the opening and which is sealed about the opening by bonding. The closure and the container provide between them at least one attachment mechanism by which the closure can be re -attached to the container over the opening to shut the opening. The surgical pack can be used by removing the closure and then removing the surgical equipment. After use, the surgical equipment is replaced in the container and the closure is re -attached over the opening to hold the replaced surgical equipment within the container. The container and used surgical equipment can then safely be disposed of.

The present invention relates to a surgical pack and in particular to a pack having a re-attachable closure so that the pack can be re-used to dispose of its original contents.

It is common to provide surgical equipment, such as surgical instruments, tools, implants and the like, in packaging which holds the surgical equipment to maintain its sterility. The surgical equipment is then transferred to the surgeon so as to maintain the sterility of the equipment so that the surgeon can use the surgical equipment. With reusable surgical equipment, the surgical equipment is simply kept after use and is then cleaned, repackaged and sterilised for re-use. However, for disposable surgical equipment, the surgical equipment needs to be disposed of. However, it may not be possible simply to dispose of the surgical equipment using normal garbage handling as the surgical equipment may be dirty or potentially dangerous.

There is limited space and time in an operating theatre and so it would be beneficial to be able to dispose of surgical equipment, whether re-useable or not, in a simple and rapid manner without increasing the complexity of the operating theatre environment or increasing clutter within the operating theatre.

The present invention addresses such problems by providing a surgical pack which can be re-used to facilitate safe disposal of at least some of the surgical equipment originally present in the pack.

According to a first aspect of the invention there is provided a surgical pack, comprising: a container having an opening; at least one item of surgical equipment received by the container; and a closure which shuts the opening and which is sealed about the opening of the container by bonding, wherein the closure and the container provide between them at least one attachment mechanism by which the closure can be re-attached to the container over the opening to shut the opening.

As the closure can be re-attached to the container, the container can be re-used, for example to more safely dispose of some of the original contents of the container after their use. Hence, no ancillary waste disposal facilities need to be provided in the surgical environment as the original packaging, which is present anyway, can be re-used for waste disposal without requiring modification of existing waste management. The container helps to protect the user and parts of the conventional waste disposal process from the contents being disposed of and the re-attachable lid helps to retain the contents within the container.

The closure can be bonded, adhered or stuck to the container so as to seal the container using a number of different non-mechanical attachment mechanisms. For example, the closure can be heat bonded or welded or an adhesive can be used. A part or all of the closure can be sealed to the container by bonding. A non-bonded part of the closure can be permanently attached to the container, for example as a living hinge or similar.

The attachment mechanism can be provided in a number of different ways. For example, the attachment mechanism can be a touch or contact fastener. For example the attachment mechanism can use bonding. Various types of bonding can be used. For example the bonding can be provided by an adhesive material. The attachment mechanism can include one or more areas of adhesive. The one or more areas of adhesive can be provided on the container and/or on the closure. The one or more areas of adhesive can be provided around the opening. The adhesive can be a contact adhesive or on surface of a piece or pieces of double sided adhesive tape. Other types of adhesives can be used such as photo-reactive adhesives, chemically reactive adhesives.

The attachment mechanism can comprise one or more regions of hook and loop fastener material. The one or more regions of hook and loop fastener can be provided on the container and/or on the closure. The one or more regions of areas of hook and loop fastener can be provided around the opening.

Other types of non-mechanical attachment mechanisms can also be used. For example, the attachment mechanism may be magnetic, may use static electricity (similarly to cling film) or may use surface tension.

The attachment mechanism can be one or a plurality of mechanical fastener mechanisms. A part of the or each mechanical fastener mechanism can be provided by a part of the container and/or a part of the closure. The attachment mechanisms can be provided around the opening. Each attachment mechanism can comprise a male component and a female component. The male and female components can be engageable or interlockable so as to fasten together. The or each female component can be an aperture, slit or hole. The or each male component can be a projection. The male and female components can be releasably engageable or interlockable so as to releasably fasten together. The or each male component can be provided by a part of the container and the or each female component can be provided by a part of the closure and vice versa.

The or each fastener mechanism can include or wholly comprise an elastic or resiliently deformable part.

Each fastener mechanism can be a releasable fastener mechanism so that the closure can be removed from and further re-attached to the container.

The closure can include a sealing portion which seals about the opening. The closure can be sealed to the container by a number of mechanisms. The closure may be heat sealed or sealed by an adhesive or other form of bonding. Preferably the closure is sealed to the container by a thermal bond.

The closure can include a peripheral portion toward the edge of the closure from the sealing portion. The peripheral portion of the closure can includes at least a part of the attachment mechanism or mechanisms.

The closure can include one or more tabs adjacent a closure part of the attachment mechanism and manipulable by a user to help re-attach the closure and/or operate the or each attachment mechanism.

The pack can have a plurality of attachment mechanisms. A peripheral part of the closure can include a plurality of apertures spaced around the closure. The container can include a plurality of male parts spaced around the opening of the container. The positions of the female and male parts can coincide or be in registration to allow each male part to engage or interlock with a respective female part.

The closure can comprise or include a lid, a base or a wall. The container can include one or more side walls. When the closure comprises a lid, a component of the attachment mechanism can be provided by one or more of the side walls of the container. Each side wall of the container can include at least one component of a one of the attachment mechanisms.

The closure can be entirely removable from the container. The closure can be permanently attached to the container while moveable to expose the opening sufficient to access the interior of the container. The closure can be attached to the container by one or more hinge portions.

The closure can be a sheet or film of material. The closure can be a single layer or a multi-layer film. The film can have a 2-layer construction. The closure can be made of a flexible material. The closure can be made of a plastic and/or a polymeric material. The closure can be a thermoplastic material. Various types of material can be used, including polycarbonates, polyethylene, polyethylene terephthalate, polyurethane, thermoplastic polyurethane, polyvinyl chloride, polyester or polypropylene. The closure can have a layer of adhesive material as a bottom layer.

A portion of the container can be deformable, preferably resiliently deformable, to allow the attachment mechanism to operate. The closure and/or the container can be resiliently deformable to allow the attachment mechanism to be engaged. The material and/or the structure of the closure and/or the container can make them or it resiliently deformable.

The attachment mechanism can include one or a plurality of container components. The or each container component can be provided as an integral or unitary part of the container. The or each container component can be a molded or otherwise formed part of the container.

The container can be a formed, vacuum formed, thermo-formed or molded article.

The container can be made of a thermoformable thermoplastic material, such as a thermoplastic polymer. The container can be made from a formable, vacuum formable, thermo-formable, or moldable material. Suitable materials for the container include acrylonitrile butadiene styrene (ABS), polypropylene (PP), polystyrene, high impact polystyrene (HIPS), polyethylene terephthalate (PET), Polyethylene terephthalate glycol (PETG), crystallised polyethylene terephthalate (CPET), styrene acrylonitrile (SAN), polyvinyl chloride (PVC) or polymethylmethacrylate (PMMA).

The surgical pack can include at least one item of disposable surgical equipment. The surgical pack can include non-disposable and disposable items of surgical equipment. The surgical pack can include a plurality of items of surgical equipment. The surgical pack can include a plurality of items of disposable surgical equipment. The surgical equipment can include orthopaedic surgical equipment. The surgical equipment can include tools, instruments, cutting blocks, guides, trial implants, implants, fixings and ancillary components, parts, fixtures and fittings thereof.

The container can include one or a plurality of formations toward a floor of the container. The formation or formations can be positioned, sized, shaped and/or otherwise configured to define one or a plurality of locations each for receiving a specific piece of surgical equipment. The or each formation can be formed in the floor of the container. The or each formation can be formed in one or more separate parts which are located on the floor of the container. The separate part can be in the form of a tray. This can assist with checking and counting parts out of the patient to ensure that nothing is left behind during use of the pack.

According to a second aspect of the invention there is provided a method of providing a surgical pack, the surgical pack comprising a container holding at least one item of surgical equipment and a closure sealed about the opening by bonding, the method comprising: locating at least one item of surgical equipment in a container via an opening of the container; shutting the opening by sealing a closure over the opening by bonding the closure to the container, wherein the closure and container provide between them at least one attachment mechanism by which the closure can be re-attached to the container over the opening to shut the opening.

As the closure can be re-attached to the container, the container can be re-used to dispose of surgical waste or disposable items.

The method can further comprise manufacturing the container using a molding or forming process. One or more container components of one or more attachment mechanisms can be formed as part of the container by the molding or forming process.

The closure can be bonded to the container by a heat sealing process. Other non-mechanical bonding processes can be used, such as welding or an adhesive can be used.

The method can further comprise sterilising the surgical pack. The method can further comprise delivering the sterilised surgical pack to a medical facility.

According to a third aspect of the invention there is provided a method of using a surgical pack comprising a container holding at least one item of surgical equipment and a closure sealed about an opening of the container by bonding, the method comprising: at least partially removing the sealed closure to provide access to the at least one item of surgical equipment via an opening of the container; removing the at least one item of surgical equipment; replacing the at least one item of surgical equipment in the container; an re-attaching the closure to the container over the opening to hold the replaced surgical equipment within the container using at least one attachment mechanism provided by the closure and the container.

Hence, the container can be re-used to facilitate disposal of at least one item of surgical equipment originally supplied in the pack.

The method can further comprise disposing of the container holding the replaced item of surgical equipment and the re-attached closure.

Either of the method aspects of the invention can be applied to any of the surgical pack aspects of the invention.

Embodiments of the invention will now be described in detail, by way of example only, and with reference to the accompanying drawings, in which:

FIG. 1 shows a plan view of a first embodiment of a surgical pack according to the invention;

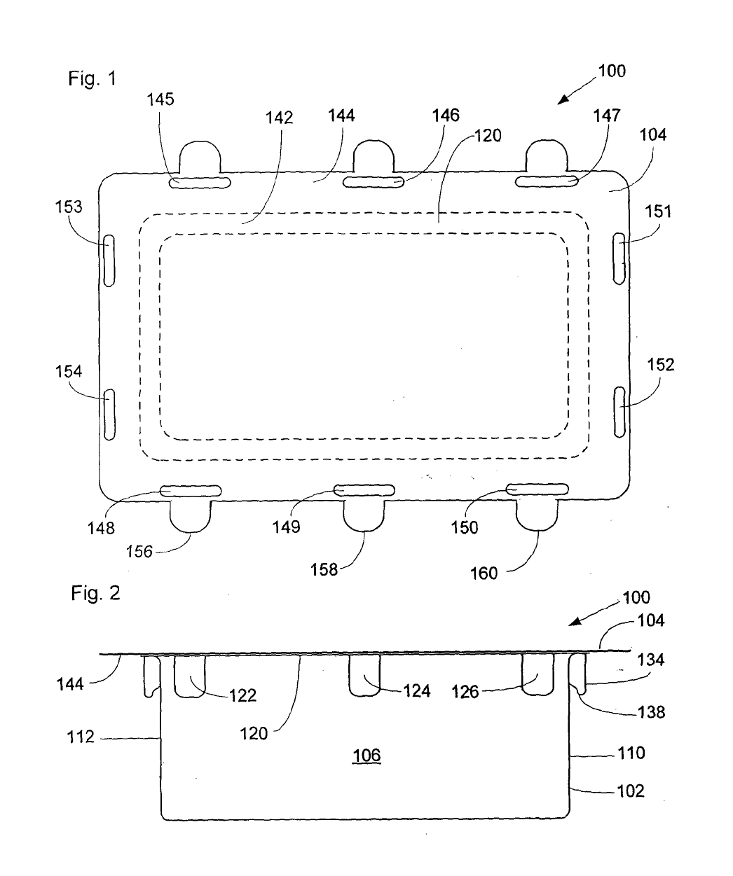

FIG. 2 shows a side view of the surgical pack shown in FIG. 1;

FIG. 3 shows a perspective view of the surgical pack shown in FIGS. 1 and 2 illustrating removal of the lid during use of the surgical pack;

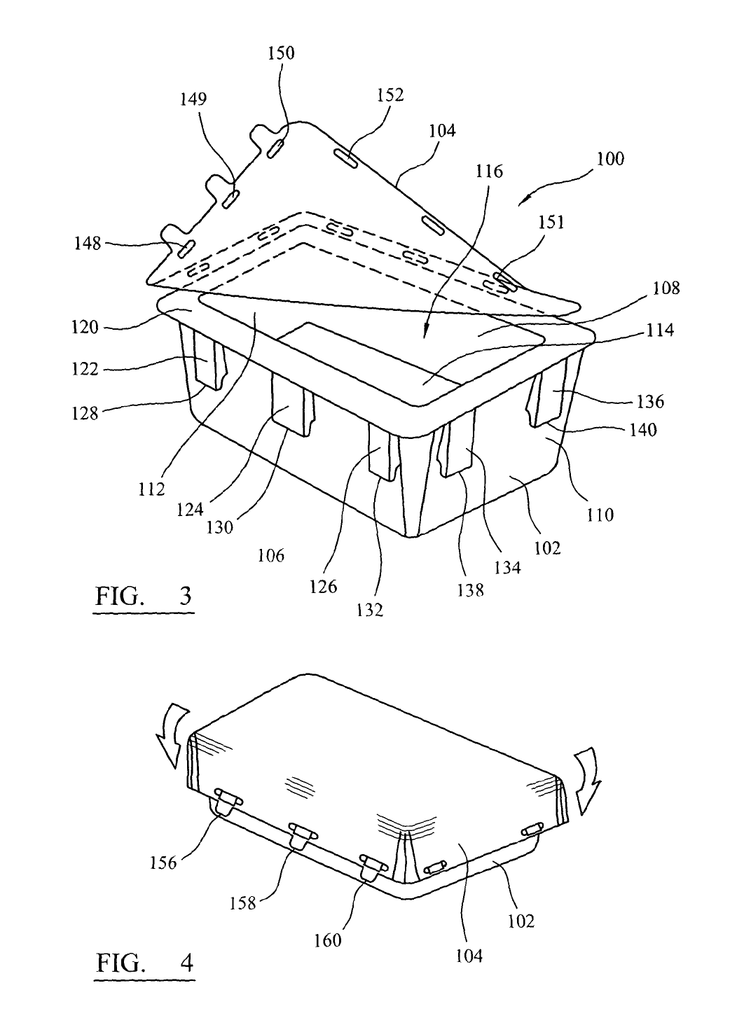

FIG. 4 shows a perspective view of the surgical pack shown in FIGS. 1 and 2 illustrating re-attachment of the lid during use of the surgical pack;

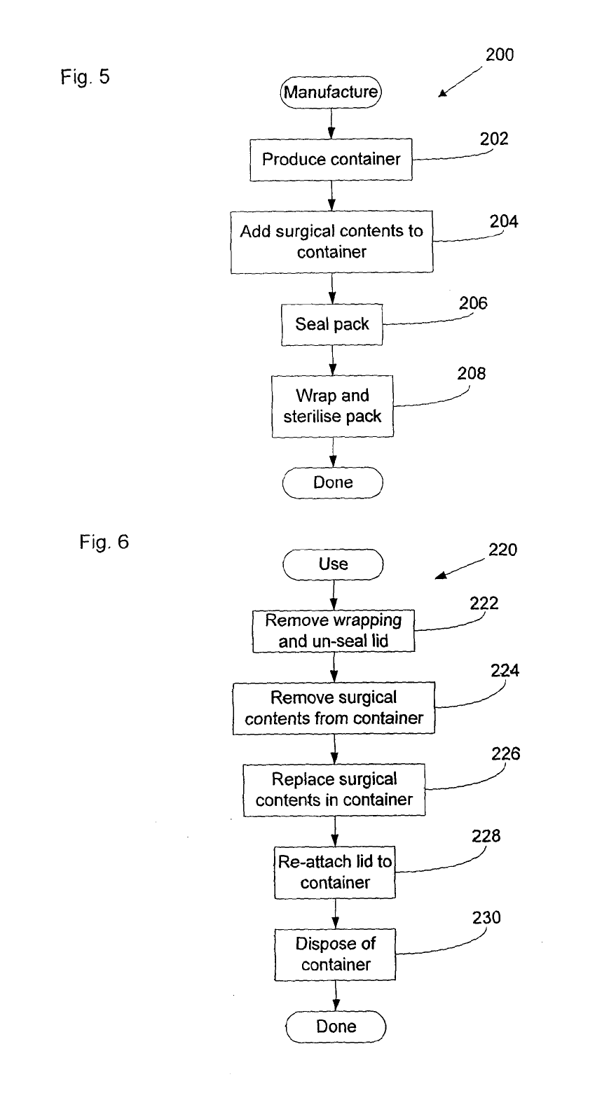

FIG. 5 shows a flow chart illustrating a method of providing a surgical pack according to the invention;

FIG. 6 shows a flow chart illustrating a method of using a surgical pack according to the invention;

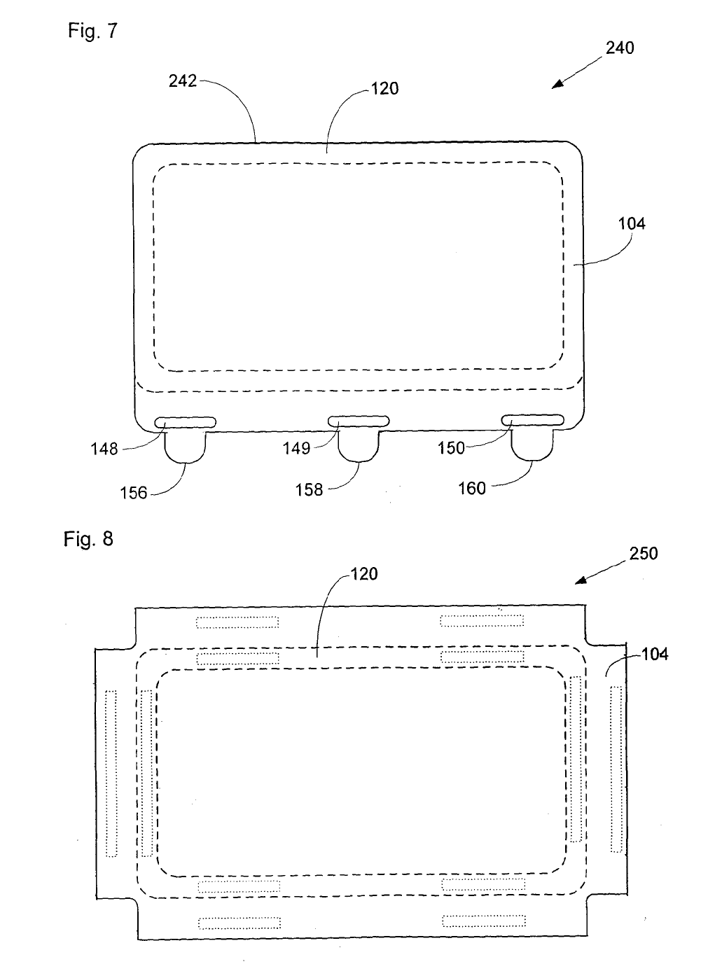

FIG. 7 shows a plan view of a second embodiment of a surgical pack according to the invention;

FIG. 8 shows a plan view of a third embodiment of a surgical pack according to the invention;

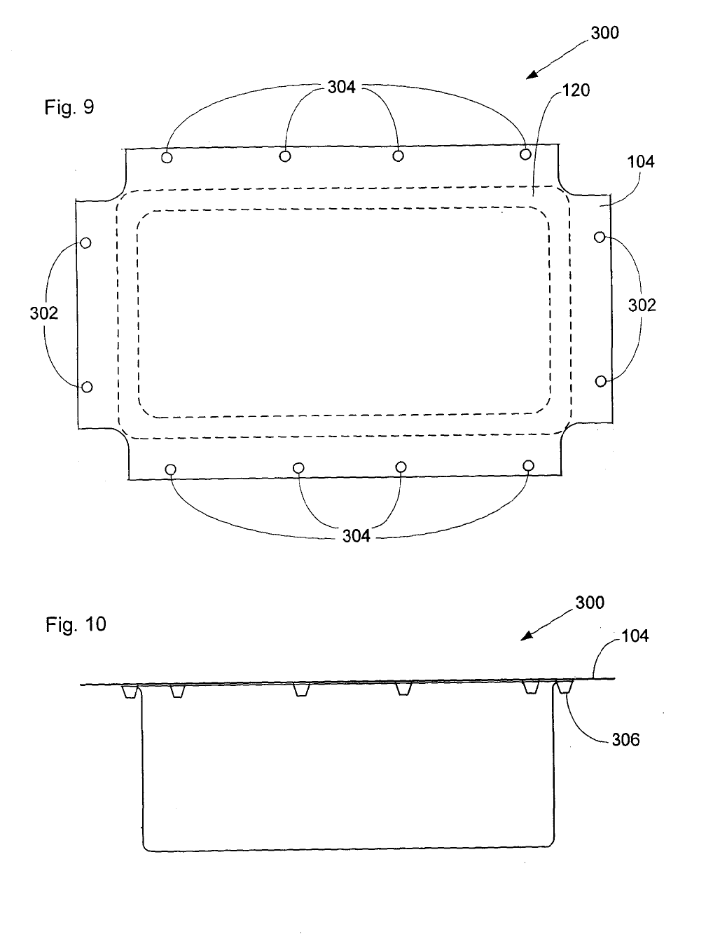

FIG. 9 shows a plan view of a fourth embodiment of a surgical pack according to the invention;

FIG. 10 shows a side view of the surgical pack shown in FIG. 9;

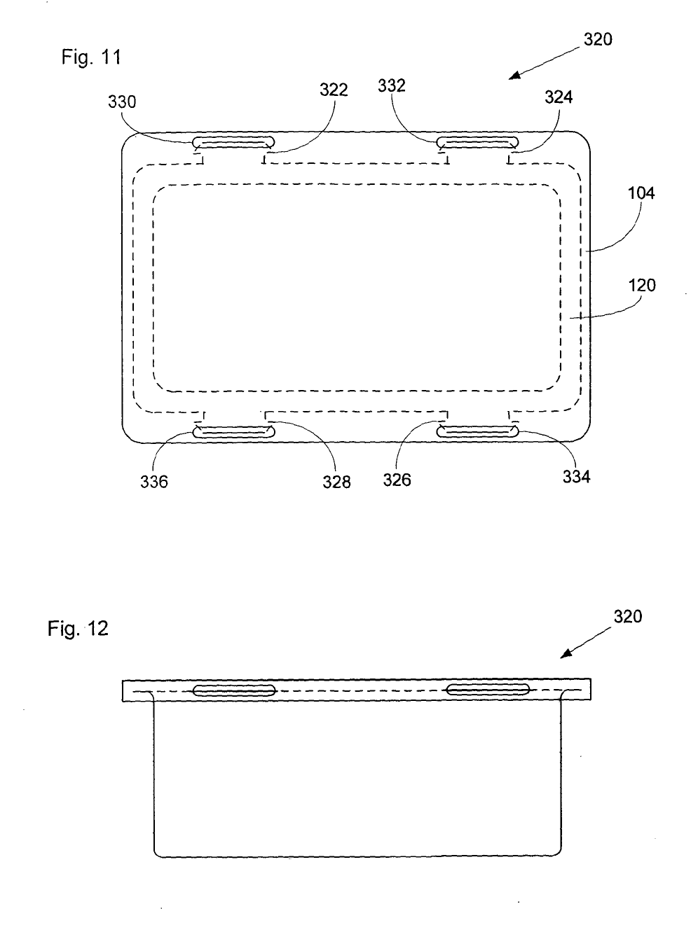

FIG. 11 shows a plan view of a fifth embodiment of a surgical pack according to the invention; and

FIG. 12 shows a side view of the surgical pack shown in FIG. 11 with the lid re-attached.

Similar features in the different Figures share common reference numerals unless indicated otherwise.

With reference to FIGS. 1 to 3, there is shown a surgical pack 100 according to a first embodiment of the invention. The surgical pack generally includes a container 102 and a closure 104.

As best illustrated in FIG. 3, the container is in the form of a generally rectangular hollow box like shape having opposed side walls 106, 108 and opposed end walls 110, 112 and a base 114. The side and end walls define an opening 116. A lip or flange 120 (shown in broken lines in FIG. 1) extends around opening 116 and extends generally perpendicularly from the side and end walls.

The side and end walls each bear container components of an attachment mechanism for re-attaching closure 104 as will be described in greater detail below. More specifically, each side wall bears three formations 122, 124, 126 each having a downward projecting tongue 128, 130, 132. Similarly, each end wall bears two similar formations 134, 136 similarly each having a generally downward projecting tongue 138, 140.

The container 102 is made from a sheet of thermo-formed material and the container attachment mechanism components are integrally formed with the main structure of the container when the container is formed. Suitable materials for the container include polystyrene, polypropylene and high density polyethylene (HDPE). The container can also be made from ABS, PET, SAN, PVC, HIPS, PETG, CPET or PMMA, amongst others. The container can be made by other manufacturing processes such as vacuum forming, injection molding, blow molding and draping.

The container 100 also includes closure 104 which, in the illustrated embodiment, takes the form generally of a lid. Lid 104 shuts opening 116 of the container and is sealed to the lip 120 of the container all the way around opening 116. The lid is heat sealed to the lip 120 of the container. In other embodiments, depending on the material of the lid, the lid may be sealed to the container about the container's opening using an adhesive or other sealing technology. The lid is in the form of a flexible sheet of material. A suitable material for the lid includes a non-woven high-density polyethylene material such as Tyvek as provided by DuPont (Tyvek is a registered trade mark in some countries). Other suitable materials for the lid include other plastics and polymers, including, for example, PC, PE, PET, PU, TPU, PVC, Polyester and PP. The lid can be made of a 2-layer film including a layer of PET and an adhesive or sealant layer on the bottom. A suitable material for the lid is the RPP#21-1260 film as provided by Rollprint Packaging Products, Inc. of Illinois, USA. Other sealant materials would include a polybutene blend (e.g. polybutene-LDPE-EVA), polypropylene blends and polyethylene blends.

As best illustrated in FIG. 1, the lid includes a portion 142 which seals against the lip 120 of the container and an outer peripheral portion 144 extending around the container and overhanging the lip of the container, as can be seen in FIG. 2. Three slots 145, 146, 147 and 148, 149 and 150 are provided toward each side edge of the lid. Two slots 151, 152 and 153, 154 are provided toward each end edge of the lid. The slots are positioned such that when the lid is sealed to the container, the slots are generally adjacent the container attachment mechanism components. The slots in the lid provide a closure attachment mechanism component which can be used to reattach the lid to the container by interlocking the respective tongues and slots as will be described in greater detail below.

As also shown in FIGS. 1 and 3, the lid includes three tabs extending from each side edge 156, 158, 160. The tabs can be grasped in use by a user to facilitate re-attachment of the lid to the container as described in greater detail below. In other embodiments, tabs can also be provided adjacent the end slots 151, 152, 153 and 154.

One or more items of surgical equipment is located within the container. The surgical equipment may include surgical instruments, tools, implants or similar. At least one of the items of surgical equipment may be disposable in that it may be intended for a single use such that it is disposed of after use during surgery. Commonly, a tray holding the one or more items or surgical equipment may be located within the cavity defined by the container.

Before describing use of the surgical pack, the method of manufacture of the surgical pack will be described with reference to FIG. 5.

FIG. 5 shows a process flow chart illustrating a method of manufacture of a surgical pack according to the invention. The method of manufacture 200 begins at step 202 by producing the container 102 of the surgical pack. The container can be produced by a moulding or forming process in which the container and the container attachment mechanism components are integrally formed as a single unitary part. The container 102 can be thermo-formed from a sheet of thermo-formable material such a polystyrene, polypropylene or HDPE. In alternative embodiments, the container can be vacuum formed from a suitable material. Once the container has been formed, the one or more items of surgical equipment are located in the container via its opening 116. The items or surgical equipment may be located in a tray or other support structure within the container 102. Several trays or surgical equipment may be stacked or placed side by side within container 102. Once the intended surgical contents of the container are present in the container, the lid 104 is located over the opening 116 and sealed about the opening so as to shut the opening at step 206. The lid 104 may be heat sealed to the lip 120 of the container. In other embodiments, the lid may be bonded using other sealing mechanisms, such as an adhesive.

Once the lid has been sealed to the container, any other layers or wrapping material and further packaging can be added to the surgical pack. The surgical pack may then be sterilised, for example by gamma irradiation, either at the manufacturing facility or at a separate sterilisation facility. The sterilised pack may then be stored or delivered to a medical facility for storage and subsequent use.

FIG. 6 shows a process flow chart illustrating a method of use 220 of the surgical pack 100 according to a further aspect of the invention. The sterilised surgical pack is located in the operating theatre and at step 222, any wrapping and/or other packaging, is removed from the surgical pack 100. The surgical pack is passed by a “dirty” nurse to a “sterile” nurse who then removes the lid 104 by peeling it away from the container 102. At step 224 a known aseptic transfer technique is used to remove the items of surgical equipment from the container, including any trays or other supports holding the items of surgical equipment. The opened surgical pack is then retained in the operating theatre.

The items of surgical equipment are then used during the surgical procedure by the surgeon. After use, or at the end of the surgical procedure, at least one of the items of surgical equipment is placed back in the container 102 at step 226. Other items of surgical equipment not originally present in the surgical container may also be placed in the surgical container after use. Because of the nature of the material of which the surgical container is manufactured, it is not suitable for disposing of genuine sharps, such as blades or syringes, but is suitable for disposing of other items of surgical equipment which may still give rise to the danger of puncture wounds or which are simply contaminated with bodily materials. However, the container could be used to hold sharps before the sharps are placed in a reinforced sharps container for disposal. It is ensured that the surgical equipment placed in the container lies below the plane of the lip 120 of the container.

Then the lid 104 can be reattached over the opening 116 using an attachment mechanism provided by the slits in the lid and the tongues of the container at step 228. As illustrated in FIG. 4, slits 152 and 151 are engaged with tongues 138 and 140 at a first end of the container and then slits 153 and 154 are engaged with similar tongues at the other end of the container. Then the slits along the side edges of the lid (148, 149, 150 and 145, 146, 147) are similarly engaged with respective tongues 128, 130, 132. This is accomplished by pulling the skirt of lid material extending around the lid down so as to interlock the tongue into the slit so that the lid is retained over the opening 116 of the container. This therefore helps to hold the items of surgical equipment to be disposed of within the container and prevents them escaping. The material of the container is resilient and slightly flexible and the material of the lid may also be slightly stretchy such that there is some deformation of the lid and/or the container walls possible to allow interlocking of the tongues and slits. The material at the corners of the lid may be folded slightly at the corners as illustrated in FIG. 4.

Once the lid has been reattached to the container, the container and its contents may be disposed of using the medical facilities conventional waste disposal. For example, the pack and contents may be disposed of in a sharps bin or similar.

A number of advantages arise from re-using the original packaging in which surgical equipment is provided in order to subsequently dispose of the surgical equipment. Firstly, there is no need to provide a different waste disposal facility and the already existing waste disposal facilities of a hospital or medical facility can be used. If no packaging to dispose of the surgical equipment were available, then a nurse may need to find other packaging material to wrap up contaminated equipment or to try and make safe surgical equipment which may give rise to the risk of puncture wounds or other wounds if disposed of without being wrapped. For example, there may be some risk of a blunt instrument still tearing a conventional garbage bag or sack in the absence of some form of wrapping. As the same pack which is used to deliver the sterile surgical equipment is subsequently reused to dispose of the surgical equipment, no further materials not already present in the operating theatre need to be introduced. Further, by reattaching the original lid, the contents of the container are held securely within the container and may not fall out so as to damage a garbage bag or sack. Hence, safe and prompt disposal of surgical equipment can be carried out using the surgical pack of the invention with its reattachable lid.

It will be appreciated that there are a large number of variations and modifications to the surgical pack of the invention. Some further embodiments of the surgical pack will be described below. Unless indicated otherwise, the general construction and use of the other embodiments of the surgical pack are similar to that of the first embodiment.

With reference to FIG. 7, there is shown a plan view of a second embodiment of a surgical pack 240 according to the invention. Surgical pack 240 is similar to surgical pack 100. However, surgical pack 240 includes container attachment mechanism components 122, 124, 126 and only a single side wall of the container. Further, lid 104 includes three slots 148, 149, 150 and three tabs 156, 158, 160 along only a single edge of the lid. The lid differs to that of the first embodiment in that it is permanently attached to the lip of the container by a hinge portion 242 along one of the edges of the lip 120. Hinge 242 may be provided by a separate piece of material or simply by permanently bonding lid 104 to the lip of the container so as to allow the lid 104 to pivot thereabout. The lid 104 may be sealed about the entire lip 120 or only along the end portions and side portion opposed to the side portion including the hinge.

Use of the surgical pack is similar to that of the first surgical pack. Initially, the lid 104 is sealed to the container and is peeled away from the container but not totally removed from the container as it is attached by hinge portion 242. After use, one or more items of surgical equipment are retained to the container and then tabs 156, 158, 160 are used to pull the slots 148, 149, 150 over the respective tongues of the container so as to reattach lid 104 over the opening 116 of the container.

FIG. 8 shows a third embodiment 250 of the surgical pack. The third embodiment differs from the first and second embodiments in that it includes a non-mechanical attachment mechanism by which the lid can be reattached to the container. In a first version, patches of adhesive material are provided on the underside surface of the peripheral skirt portion of the lid and/or on the underside of the lip 120 of the container. For example, two separate regions of adhesive material can be provided on each side portion of the lid and a single portion of adhesive material on each end portion of the lid. As illustrated in FIG. 8, the corner portions of the peripheral overhanging skirt portion of the lid has been removed so as to make it easier to fold the lid down and over the lip 120 of the container. The portions of adhesive material may be a contact adhesive or may be regions of double sided tape including a strip of release material removable from the tape to allow re-attachment of the lid by folding the peripheral tab portions of the lid over the lip 120 of the container so as to adhere to the underside of the lip.

In another version of the third embodiment, the regions of adhesive material are provided on the underside of the lip 120 and not on the underside of the lid 104. In use, the peripheral portions of the lid are folded over the lip and pressed against the regions of adhesive material on the underside of the lip so as to re-attach the lid to the container.

In a yet further version, instead of using an adhesive material, regions of hook and eye fastener, or similar, material are used to provide the attachment mechanism. Suitable material is provided under the name Velcro (Velcro is a trade mark registered in some countries). Regions or hook or eye material are provided on the underside of the lip 120 of the container and the other of the hook or eye material is provided on the underside of the peripheral part of the lid 104. Hence, in use, the peripheral portions of the lid are folded over the lip 120 so as to engage the hook and eye materials to re-attach the lid to the container.

FIGS. 9 and 10 show plan and side views of a fourth embodiment of the surgical pack 300 according to the invention. In this embodiment, again, the corner portions of the lid have been cut away such that the peripheral portions of the lid form pairs of opposed flaps. The end flaps include two circular apertures and the side flaps each include four circular apertures 304. As illustrated in FIG. 10, a plurality of similar members 306 project downwardly from the underside of lip 120. Four members are provided on each side of the container and two members on each end of the container. Each member 306 is in the form of a frusto-conical member having a largest diameter similar to the diameter of the circular apertures in the lid. The frusto-conical member is formed in the underside of the lip 120 during formation of the container. The members 306 have a generally inverted circular conical shape with a flat free end and are positioned on the lip adjacent to their corresponding respective aperture in the lid when initially sealed to the container.

In use, in order to re-attach lid 104 to the container, the flaps of the lid are folded over the lip and the heads of the projections 306 are pushed through the corresponding apertures in the flaps of the lid so as to interlock the projections and apertures. Hence, the lid can be securely re-attached to the container to prevent surgical items falling out of the container and therefore allowing safe disposal.

FIGS. 11 and 12 show plan and side views of a fifth embodiment of a surgical pack 320 according to the invention. As illustrated in FIG. 11, the lip 120 of the container includes two container attachment mechanism components extending from each side 322, 324, 326, 328 (illustrated in dashed lines). Each component has the general form of a body bearing a generally trapezoid shaped head and is an integrally molded part of the lip 120.

The lid 104 includes two generally slot shaped apertures 330, 332, 334, 336 positioned in the peripheral skirt portion of the lid 104 generally adjacent the head of the container attachment mechanism component. Slots 330, 332, 334, 336 provide a closure attachment mechanism component which in use interacts with the container attachment mechanism component to re-attach the lid.

FIG. 12 shows a side view of the surgical pack 320 after the lid has been re-attached. In use, the lid 104 can be re-attached to the container holding the surgical items to be disposed of by folding the side portions of the lid 104 and engaging the heads of the container components into the slots in the lid. The slots in the lid have a similar lateral dimension to the size of the head of the container component and so are generally retained by the head of the container component by interlocking therewith.

It will be appreciated that in other embodiments, a fewer or greater number of closure slots and container components can be provided. Also, slots and container components can be provided on the ends of the surgical pack.

The placing or replacing of instruments back in the pack offers the opportunity to “count out” the instruments from the surgical site so as to help ensure that all instruments are accounted for toward the end of the surgical procedure. An embodiment of the invention includes providing a plurality of formations in the bottom of the container positioned and configured to provide specific locations for each instrument. The formations can be provided as an integral part of the container by being molded or otherwise formed in the floor of the container during manufacture of the package. Alternatively, the formations can be provided as a separate part, like a tray, which sits in the floor of the package. This will assist with checking and counting parts out of the patient to ensure that nothing is left behind.

It will be appreciated that a wide number of variations and modifications are possible to the specific embodiments described above. Further, features of some of the embodiments can be used in combinations with the features of the other embodiments. Further, the number of specific features of each component can be varied depending on the size, geometry and other usability requirements of any particular surgical pack. Therefore, various modifications and alternatives will be apparent to a person of ordinary skill in the art from the discussion of the invention presented above.

1. A surgical pack, comprising:
a container having an opening;
at least one item of surgical equipment received by the container; and
a closure which shuts the opening and which is sealed about the opening of the container by bonding, wherein the closure and the container provide between them at least one attachment mechanism by which the closure can be re-attached to the container over the opening to shut the opening.

2. The pack of claim 1, wherein the attachment mechanism comprises one or more areas of adhesive.

3. The pack of claim 1, wherein the attachment mechanism comprises one or more regions of hook and loop fastener material.

4. The pack of claim 1, wherein the attachment mechanism is one or more mechanical mechanisms.

5. The pack of claim 4, wherein each attachment mechanism comprises a male component and a female component and wherein the male and female components are interlockable.

6. The pack of claim 5, wherein the male components are provided by a part of the container and the female components are provided by a part of the closure.

7. The pack of claim 1, wherein the closure includes a sealing portion which seals about the opening and the closure includes a peripheral portion toward the edge of the closure from the sealing portion and wherein the peripheral portion includes at least a part of the attachment mechanism.

8. The pack of claim 1, wherein the closure includes one or more tabs adjacent a closure part of the attachment mechanism and manipulable by a user to help re-attach the closure.

9. The pack of claim 6, wherein the pack has a plurality of attachment mechanisms and a peripheral part of the closure includes a plurality of apertures spaced around the closure and the container includes a plurality of male parts spaced around the opening of the container and wherein the positions of the female and male parts coincide to allow each male part to interlock with a respective female part.

10. The pack of claim 1, wherein the closure comprises a lid, the container includes side walls and wherein a component of the attachment mechanism is provided by at least one of the side walls of the container.

11. The pack of claim 1, wherein the closure is attached to the container by a hinge portion.

12. The pack of claim 1, wherein the closure is made of a flexible material.

13. The pack of claim 1, wherein the closure and/or the container are resiliently deformable to allow the attachment mechanism to be engaged.

14. The pack of claim 1, wherein the attachment mechanism includes a container component and wherein the container component is provided as an integral part of the container.

15. The pack of claim 14, wherein the container component is a molded part of the container.

16. The pack of claim 1, wherein the container includes a plurality of formations toward a floor of the container which are positioned and configured to define one or a plurality of locations each for receiving a specific piece of surgical equipment.

17. A method of providing a surgical pack, the surgical pack comprising a container holding at least one item of surgical equipment and a closure sealed about the opening by bonding, the method comprising:
locating at least one item of surgical equipment in a container via an opening of the container;
shutting the opening by sealing a closure over the opening by bonding the closure to the container, wherein the closure and container provide between them at least one attachment mechanism by which the closure can be re-attached to the container over the opening to shut the opening.

18. The method of claim 17, and further comprising:
manufacturing the container using a molding process and wherein a container component of the attachment mechanism is formed as part of the container by the molding process.

19. The method of claim 17, wherein the closure is sealed over the opening by a heat sealing process.

20. The method of claim 17, and further comprising sterilising the surgical pack.

21-23. (canceled)
